(19) United States
(12) Patent Application Publication
(10) Pub. No.: US 2007/0142920 A1
(43) Pub. Date: Jun. 21, 2007

(54) Metatarsal implant

(76) Inventor: Willard J. Niemi, Cary, NC (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/311,987

(22) Filed: Dec. 20, 2005

(51) Int. Cl. A61F 2/42; A61B 17/80

(52) U.S. Cl. 623/21.19; 606/69

(57) ABSTRACT

An implant that may be surgically positioned proximate a metatarsal may include a body having an exterior portion and two interior (or center) portions positioned on opposite sides of the body. Apertures suitable for accepting sutures to secure the implant in position may be provided along a perimeter (or margin) of the exterior portion of the body.

### BACKGROUND OF THE INVENTION

1. Field of the Invention

The present invention is directed to an implant for use in positioning/ distributing pressure the metatarsal head or metatarsophalangeal joint. More specifically, the invention is directed to an implant that may be positioned proximate a metatarsal of the foot, e.g., disposed under the condyles of the metatarsal head, to thereby properly padding, cushioning or distributing force in a particular metatarsal and better distribute the weight of the user relative to the other metatarsals and relieve pressure and hyperkoratotic lesions.

2. Reference to Related Art

Maladies normally associated with the improper alignment of one or more metatarsals of the foot include plantar metatarsal head calluses, atrophy of the plantar fat pad, and a planar flexed metatarsal. In severe cases of head calluses, metatarsal misalignment may result in an IPK developing on the planar aspect of the foot. Under typical conditions, orthotics or shoe style changes may relieve this problem. However, where the problem persists surgical intervention may be necessary. In other cases, such as where the metatarsal may be too low or too poorly positioned for orthotics to work, surgery may be the only proper means to correct the problem.

Metatarsal-related surgery requires cutting a metatarsal bone and correcting the bone length such that the patient's body weight and pressure are more evenly distributed across the foot. A common type of metatarsal surgery for correcting maladies related to the second, third and fourth metatarsal is an osteotomy. An oblique osteotomy is commonly used for surgery on the smaller fifth metatarsal or an ostectomy of the condyles may also be performed. Following surgery, a patient may expect a recover period of six to eight weeks during which he or she will be required to wear a walking cast. However, notwithstanding the availability of surgery, it is recognized that such procedures often fail due to either a return of a lesion (caused by a failure to properly elevate the bone) or a transfer of the lesion (caused by a shifting of the patient's weight to an adjacent metatarsal).

Atrophy of the plantar fat pad is caused by a thinning or compression of the adipose tissue under the plantar region of the foot. Such thinning may lead to chronic inflammation and pain directed under the affected bone or joint. The majority of patients afflicted with plantar fat pad atrophy are women between the ages of 30 and 60 who wear dress shoes. Typically, orthotics fit poorly in women's dress shoes. Therefore, the most conservative methods of treatment are not available to a majority of the patient population.

Other methods to correct various forms of foot maladies include U.S. Pat. No. 5,497,789, which was directed to a foot protector that incorporated a viscoelastic gel and functioned as a pad or medicating device for corns, hammertoes, bunions or blisters. The protector was constructed of a noncompressible thermoplastic viscoelastic gel or material that was impregnated directly onto an elastic fabric carrier. The protector was further described as being operable to internally absorb friction or horizontal shearing forces as well as having certain medicinal properties.

Another reference is U.S. Patent Application Publication No. 2003/0163161 A1, which disclosed a bone anchor protective cover. Specifically, the reference disclosed a low profile, bioabsorbable bone anchor cover. The cover was used to isolate bone anchors and reduce accidents caused by the tip or edges of the anchor prior to insertion into a target site.

Also of interest is U.S. Pat. No. 6,017,366, which disclosed a reabsorbable interpositional arthroplasty implant. Specifically, the reference concerned a reabsorbable implant that operated to fill a void between two adjacent bone ends and thereby provide a cushion between those ends. The implant also prevented impingement of the ends and provided time for tissue to infiltrate into the space occupied by the implant.

A further reference is U.S. Patent Application Publication No. 2004/0247641 A1, which was directed to an interpositional arthroplasty system and method. Specifically, this reference disclosed preparations for use with orthopedic joints. The preparation may be prepared and cured ex vivo and then fitted and secured at the joint site. The preformed components may be sufficiently pliant to permit insertion through a minimally invasive portal, yet resilient enough to substantially assume or tend towards the desired form in vivo with additional forming there as needed.

Finally, solid silicone, which is solid yet flexible and durable, has been in use as a material for facial implants since about 1956. Silicone is useful as an implant material since it can be manufactured in different durometer grades such that the implant properly enhances the overlying structure. For example, the teardrop shaped standard SM-01 malar implant, of the type manufactured by Surgiform Plastic and Cosmetic Surgery Products, is typical of commercially available silicone facial implants.

### SUMMARY OF THE INVENTION

The present invention concerns an implant that may be surgically positioned proximate a metatarsal. The implant may include a body having an exterior portion and two interior (or center) portions positioned on opposite sides of the body. Apertures suitable for accepting sutures to secure the implant in position may be provided along a perimeter (or margin) of the exterior portion of the body.

Using interoperative x-ray the implant may be inserted into a canal between the plantar fat layer and the tendon sheath through an incision located at the distal digital sulcus and behind the metatarsal head. Location of the incision at the digital sulcus and behind the metatarsal may have the advantage of avoiding the need to make a weight-bearing incision. Further, the implant may be located under the metatarsal head such that an interior portion 18 of the implant 10 is disposed under the condyles (not shown) of a metatarsal head.

### BRIEF DESCRIPTION OF THE DRAWINGS

Reference will now be made to the associated drawings wherein like reference numerals refer to like parts throughout and wherein:

FIG. 1 is a top planar view showing a metatarsal implant (in phantom) constructed in accord with the present invention positioned proximate the second metatarsal with a recessed center portion of the implant disposed under the condyles of the metatarsal head;

FIG. 2 is a side cut-away planar view showing the implant of FIG. 1 positioned relative to the second metatarsal;

FIG. 3 is a top planar view of a metatarsal implant of FIG. 1;

FIG. 4 is a side cut-away view of a metatarsal implant taken along line 4-4 of FIG. 3;

FIG. 5 is an end view of the metatarsal implant of FIG. 3; and

FIG. 6 is a side view of the metatarsal implant of FIG. 3.

### DETAILED DESCRIPTION

An implant that may be surgically positioned proximate a metatarsal may include a body having an exterior portion and one or more (or center) portions positioned on faces of the body. Suturing holds, or apertures suitable for accepting sutures, or attached sutures to the implant, may be provided though the body to secure the implant in position. The aperture may be provided along a perimeter (or margin) of the exterior portion of the body or at any other suitable location. The implant may be molded from solid silicone. However, other materials such as high grade non-toxicity polyvinyl chloride (PVC), expanded polytetrafluoroethylene (ePTFE), or polyethylene may also be used in the construction of the implant or a biodegradable material. The implant may be inserted into a canal between the plantar fat layer and the tendon sheath through an incision located at the distal digital sulcus and behind the metatarsal head.

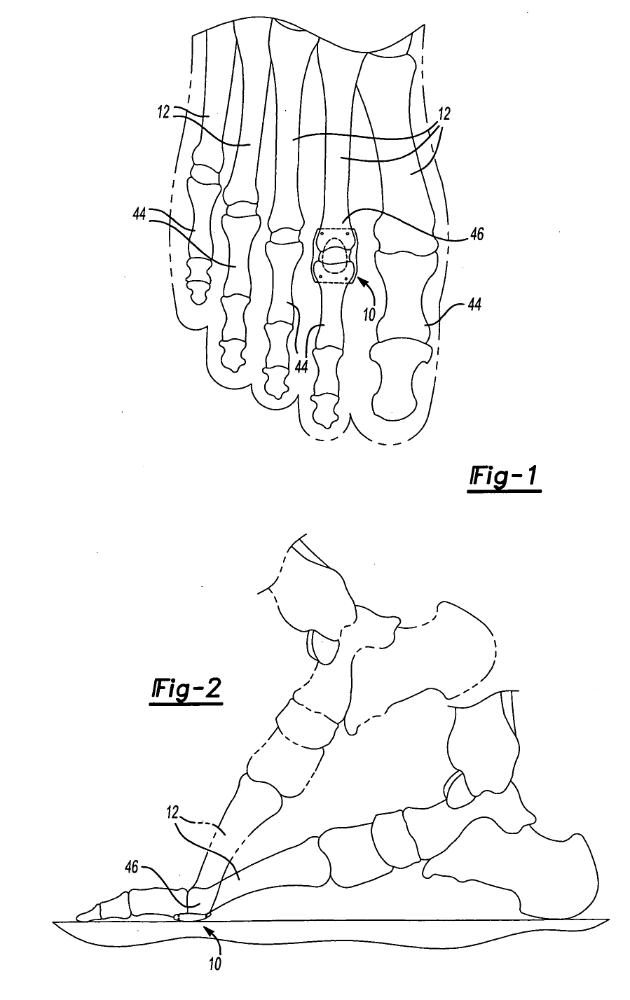

Turning now from the general description above to a description for one particular embodiment, there is as shown at FIGS. 1-6, and as best shown in FIGS. 1 and 4, an implant 10 that may be surgically positioned proximate a metatarsal 12 to thereby assist with the proper positioning to the metatarsal 12. The implant 10 may include a body 14 having an exterior portion 16 and one or more interior (or center) portions 18, 20 positioned on opposite faces 22, 24 of the body 14. Apertures 26 suitable for accepting sutures (not shown) to secure the implant 10 in position may be provided along a perimeter 28 (or margin) of the exterior portion 16 of the body 10.

Still referring to FIGS. 1-6, the exterior portion 16 of the body 14 of the implant 10 may be formed, using conventional molding techniques, as a solid, one-piece member with a substantially uniform level of hardness using implant grade solid silicone having a Shore A durometer (hardness) factor between 30 and 80. However, it will be appreciated that the material durometer may be adjusted to fit the needs of the patient. For example, the exterior portion may have a durometer factor of between 40 and 70 or between 50 and 60. The material may also have a density that permits the implant 10 to be visible to x-ray analysis. Further, other materials such as a high grade non-toxicity polyvinyl chloride (PVC), expanded polytetrafluoroethylene (ePTFE), polyethylene, Teflon, metals and metal alloys may be used, alone or in combination, in the construction of the implant 10.

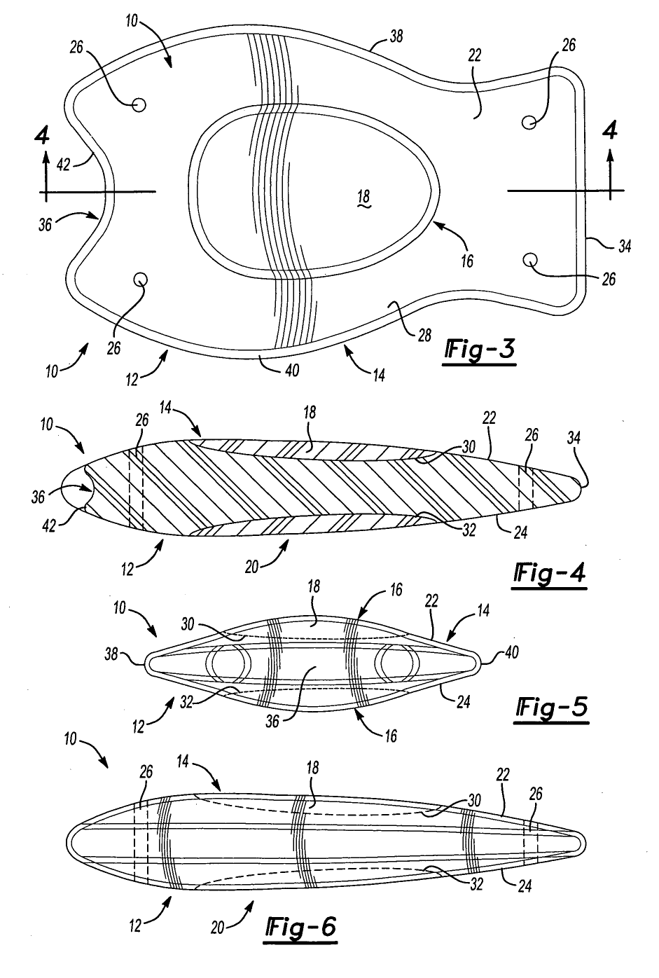

As best shown in FIGS. 3 and 4, a recess 30, 32 is defined into each face 22, 24 of the exterior portion 16 of the body 14. The exterior portion 16 may have a “teardrop” shape when viewed in profile. The perimeter 28 of the body 14 may also be rounded. Further, the ends 34, 36 and sides 38, 40 of the exterior portion 16 may be contoured according to the needs of the patient. For example, in the illustrated embodiment, one end 34 of the exterior portion 16 forms a substantially straight edge while a notch 42 is defined in the opposite end 36. The notch 42 is operable to engage or fit about the phalange 44 associate with the metatarsal 12. The sides 38, 40 of the exterior portion 16 may be likewise contoured to include areas of larger and narrower width that correspond, respectively, to the zones of greatest metatarsal 12 impact on an interior portion 18, 20 and the attachment areas for the implant 10. The apertures 26 (as discussed above) may be advantageously extended through the exterior portion 26.

Referring now to FIGS. 1 and 2-6, each interior portion 18, 20 may be bonded into one of the recesses 30, 32 defined into a face 22, 24 of the exterior portion 16 of the body 14. More particularly, each interior portion 18, 20 of the body 14 of the implant 10 may be formed and bonded to the exterior portion 16, using conventional molding techniques, as a solid, one-piece member with a substantially uniform level of hardness using implant grade silicone having a Shore A durometer (hardness) factor less that that of the exterior portion. Specifically, the interior portions 18, 20 may be formed from a solid silicone having a durometer factor between 5 and 30. However, it will be appreciated that the actual material used may be adjusted to fit the needs of the patient. For example, the interior portions 18, 20 may have a durometer factor of between 10 and 25 or between 15 and 20. Further, the interior portions 18, 20 may have a density that is less than that of the exterior portion 16 such that the interior portions 18, 20 have a visual appearance that differs from that of the exterior portion 16 of the implant 10.

Referring now to FIGS. 3-6, the body 12 of the implant 10 may have dimensions that include a length of 3.5 cm, a width of 2.5 cm, and a height (thickness) of 0.50 cm. However, similar to the issue of material selection, the final dimensions of the implant 10 may be adjusted according to the needs of the patient.

Referring now FIGS. 1 and 2, using interoperative x-ray the implant 10 may be inserted into a canal between the plantar fat layer and the tendon sheath through an incision located at the distal digital sulcus and behind the metatarsal head 46. Location of the incision at digital sulcus and behind the metatarsal 12 may have the advantage of avoiding the need to make a weight-bearing incision. Further, the implant 10 may be located under the metatarsal head 46 such that an interior portion 18 of the implant 10 is disposed under the condyles (not shown) of a metatarsal head 46.

Having thus described my invention, various other embodiments will become known to those of skill in the art that do not depart from the spirit of the present invention.

I claim:

1. An implant for use in positioning a metatarsal bone comprising:
a body having an exterior portion, an interior portion and defining apertures that extend through the body and are operable for accepting sutures to secure the implant in position proximate a metatarsal bone, the exterior portion having a pair of opposed faces, one of the faces defining a recess and the interior portion being positioned in the recess of the face.

2. The implant of claim 1, wherein the exterior portion comprises a material of a first durometer factor and the interior portion comprises a material of a second durometer factor that is less than the first durometer factor.

3. The implant of claim 2, where the first durometer factor is between 30 and 80 and the second durometer factor is between 5 and 30.

4. The implant of claim 2, where the first durometer factor is between 40 and 70 and the second durometer factor is between 10 and 25.

5. The implant of claim 1, wherein the exterior portion comprises a material of a first density and the interior portion comprises a material of a second density that is different than the density of the first density.

6. The implant of claim 5, wherein the material of a first density and the interior portion comprises a material of a second density that is different that the first density.

7. The implant of claim 6, wherein at least one of the material of a first density and the material of a second density are visible under x-ray analysis.

8. The implant of claim 1, wherein the body comprises solid silicone.

9. The implant of claim 1, wherein the body comprises of one of the group consisting of high grade non-toxicity polyvinyl chloride (PVC), expanded polytetrafluoroethylene (ePTFE), polyethylene, Teflon, a metal and a metal alloy.

10. The implant of claim 1, wherein the exterior portion comprises a perimeter and the apertures are provided along and extend through the perimeter.

11. An implant for use in positioning a metatarsal bone comprising:
a body having an exterior portion, at least two interior portions and defining apertures that extend through the body and are operable for accepting sutures to secure the implant in position proximate a metatarsal bone, the exterior portion having a pair of opposed faces, each of the faces defining a recess and each of the interior portions being positioned in one of the recesses of the faces.

12. The implant of claim 10, wherein the exterior portion comprises a material of a first durometer factor and the interior portion comprises a material of a second durometer factor that is less than the first durometer factor.

13. The implant of claim 11, where the first durometer factor is between 30 and 80 and the second durometer factor is between 5 and 30.

14. The implant of claim 11, where the first durometer factor is between 40 and 70 and the second durometer factor is between 10 and 25.

15. The implant of claim 10, wherein the exterior portion comprises a material of a first density and the interior portion comprises a material of a second density that is different than the density of the first density.

16. The implant of claim 14, wherein the material of a first density and the interior portion comprises a material of a second density that is different that the first density.

17. The implant of claim 15, wherein at least one of the material of a first density and the material of a second density are visible under x-ray analysis.

18. The implant of claim 10, wherein the body comprises solid silicone.

19. The implant of claim 10, wherein the body comprises of one of the group consisting of high grade non-toxicity polyvinyl chloride (PVC), expanded polytetrafluoroethylene (ePTFE), polyethylene, Teflon, a metal and a metal alloy.

20. An implant for use in positioning a metatarsal bone comprising:
a body having an exterior portion, at least two interior portions and defining apertures that extend through the body and are operable for accepting sutures to secure the implant in position proximate a metatarsal bone, the exterior portion including a material of a first durometer factor and having a pair of opposed faces, each of the faces defining a recess, each of the interior portions including a material of a second durometer factor and being positioned in one of the recesses of the faces.
